(19) United States
(12) Patent Application Publication
(10) Pub. No.: US 2023/0215550 A1
(43) Pub. Date: Jul. 6, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Tokyo (JP); Nobuya KITAMURA, Tokyo (JP); Tomohide HIRAGAMI, Tokyo (JP); Yasuyuki HOSONO, Tokyo (JP); Kenji NAGAMIYA, Tokyo (JP)

(21) Appl. No.: 18/058,770

(22) Filed: Nov. 25, 2022

(30) Foreign Application Priority Data
Jan. 6, 2022 (JP) 2022-001171

(51) Int. Cl. G16H 40/20; G06Q 50/26; G06Q 50/30

(52) U.S. Cl. CPC G16H 40/20; G06Q 50/265; G06Q 50/30

(57) ABSTRACT

An information processing apparatus derives an action plan of a user including a schedule and a service to be used by the user among a plurality of services related to medical care, based on symptom information and positional information related to a symptom of the user, and outputs the derived action plan.

### CROSS-REFERENCE TO RELATED APPLICATIONS

The present application claims priority under 35 U.S.C. § 119 to Japanese Patent Application No. 2022-001171 filed on Jan. 6, 2022. The above application is hereby expressly incorporated by reference, in its entirety, into the present application.

### BACKGROUND

### 1. Technical Field

The present disclosure relates to an information processing apparatus, an information processing method, and an information processing program.

### 2. Description of the Related Art

JP2021-022332A discloses a technology of determining a vehicle dispatch schedule of a vehicle equipped with a facility used for health diagnosis based on an examination item, positional information, and a desired date and time for medical care reception of a user to be diagnosed.

### SUMMARY

In recent years, online medical care in which a user such as a patient can receive medical care by a doctor by using an electronic information technology such as a computer, a camera, a video conference, and Internet communication without visiting a hospital has begun to spread. Hereinafter, a case where the user visits the hospital and receives medical care in a state of facing the doctor is referred to as face-to-face medical care to be distinguished from the online medical care. In the online medical care, since less information can be grasped by the doctor than in the face-to-face medical care, the medical care by the doctor may be difficult. Such a difficulty is caused by, for example, the fact that there are examinations that cannot be implemented in the online medical care.

In recent years, the use of a taxi dispatch service using a smartphone and a delivery service for delivering products, such as home delivery, has become widespread. Along with this, for example, in the online medical care, transmission of prescription data to a pharmacy and start of use of delivery of medicine from the pharmacy to the user has been also considered.

However, it may be difficult for the user to combine a plurality of services based on a symptom of the user to determine an appropriate action plan regarding medical care. In the technology described in JP2021-022332A, it is not considered that the user uses the plurality of services.

The present disclosure has been made in view of the above circumstances, and an object of the present disclosure is to provide an information processing apparatus, an information processing method, and an information processing program capable of presenting an appropriate action plan regarding medical care to a user.

An information processing apparatus of the present disclosure is an information processing apparatus comprising at least one processor. The processor is configured to derive an action plan of a user including a schedule and a service to be used by the user among a plurality of services related to medical care, based on symptom information and positional information related to a symptom of the user, and output the derived action plan.

An information processing method of the present disclosure is executed by a processor included in an information processing apparatus comprises deriving an action plan of a user including a schedule and a service to be used by the user among a plurality of services related to medical care, based on symptom information and positional information related to a symptom of the user, and outputting the derived action plan.

An information processing program of the present disclosure causes a processor included in an information processing apparatus to execute a process comprising deriving an action plan of a user including a schedule and a service to be used by the user among a plurality of services related to medical care, based on symptom information and positional information related to a symptom of the user, and outputting the derived action plan.

According to the present disclosure, an appropriate action plan regarding medical care can be presented to a user.

### BRIEF DESCRIPTION OF THE DRAWINGS

FIG. 1 is a block diagram illustrating an example of a configuration of an information processing system.

FIG. 2 is a block diagram illustrating an example of hardware configurations of an information processing apparatus and a user terminal.

FIG. 3 is a diagram illustrating an example of a service table.

FIG. 4 is a block diagram illustrating an example of a functional configuration of the information processing apparatus.

FIG. 5 is a diagram illustrating an example of a questionnaire display screen.

FIG. 6 is a diagram for describing derivation processing of a severity using a trained model.

FIG. 7 is a diagram illustrating an example of an action plan display screen.

FIG. 8 is a diagram for describing processing of integrating billing information of a cost.

FIG. 9 is a flowchart illustrating an example of action plan derivation processing.

FIG. 10 is a flowchart illustrating an example of cost billing processing.

### DETAILED DESCRIPTION

Hereinafter, an embodiment for implementing a disclosed technology will be described in detail with reference to the drawings.

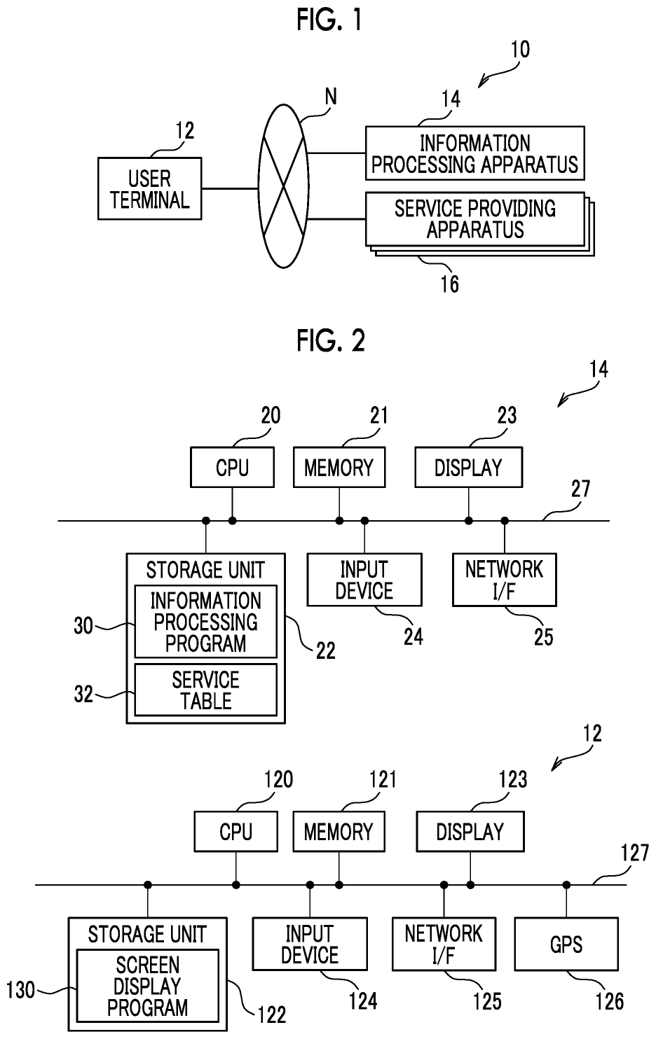

First, a configuration of an information processing system 10 according to the present embodiment will be described with reference to FIG. 1. As illustrated in FIG. 1, the information processing system 10 includes a user terminal 12 used by users of the information processing system 10, an information processing apparatus 14, and a plurality of service providing apparatuses 16. Examples of the user terminal 12 include a smartphone, a tablet-type computer, a personal computer, and the like. Examples of the information processing apparatus 14 and the service providing apparatus 16 include a server computer, a cloud server, and the like.

The user terminal 12, the information processing apparatus 14, and the service providing apparatuses 16 are connected to a network N so that data can be transmitted to and received from each other.

The service providing apparatus 16 transmits and receives information necessary for providing a service to the user to and from the information processing apparatus 14. In the present embodiment, a plurality of services related to medical care are provided to the user by using the plurality of service providing apparatuses 16. Examples of the service include a service related to medical practice, a service related to delivery for medical practice, and the like. Examples of the service related to the medical practice include an online medical care service, a face-to-face medical care service, an examination service, a providing service of medicine or the like, and the like.

The online medical care service is a service that allows the user to receive medical care by a doctor by using an electronic information technology such as a computer, a camera, a video conference, and Internet communication without visiting a hospital. The face-to-face medical care service is a service in which the doctor provides medical care to the user in a state of facing the user who visits the hospital. The face-to-face medical care service includes home-visit medical care in which the doctor visits a user's home and provides medical care to the user. The examination service is a service that performs examinations such as a specimen examination, a blood pressure examination, a hearing examination, an electrocardiogram examination, and imaging of the user by a medical imaging apparatus. Examples of the specimen examination include a blood examination, a mucous examination for a nasal cavity, an oral cavity and the pharynx, a saliva examination, a urine examination, and the like. Examples of the medical imaging apparatus include an X-ray radiographing apparatus, an ultrasonic apparatus, an endoscopic apparatus, a computed tomography (CT) apparatus, a magnetic resonance imaging (MRI) apparatus, and the like. The providing service of medicine or the like corresponds to, for example, a pharmacy, and is a service of preparing medicine based on a prescription and providing the prepared medicine. In the present embodiment, the medicine or the like is not limited to the medicine that requires a prescription, and include an examination medicine (for example, examination medicine for novel coronavirus) with which the user examines themselves and can check a result thereof) or medical supplementary products (for example, oral rehydration solutions, dressings, masks, disinfectants, diapers, and the like) that are sold in pharmacies.

Examples of the service related to the delivery for the medical practice include delivery services for medicine or the like, visit services of the examinations, transportation services for humans, and the like. Examples of the delivery service for the medicine or the like include a delivery service using an automobile, a motorcycle, a bicycle, or the like, and the like. Examples of the transportation service for the human include taxis and the like. A target person is not limited to the user, but also includes an examiner of the visit service for the examination to be described below, the doctor in the case of the home-visit medical care described above, and the like. The visit service for the examination is a service in which an examiner who can handle an examination apparatus and can implement the examination heads to the home where the user is present together with the examination apparatus by a delivery car to perform the examination. The examination apparatus includes, for example, a medical imaging apparatus such as an ultrasonic apparatus, an X-ray radiographing apparatus, and an endoscopic apparatus, an instrument for collecting or examining blood and nasal mucus, and the like. The examination includes imaging by the medical imaging apparatus, a specimen examination performed on the collected blood and nasal mucus, and the like. A delivery destination of the visit service for the examination is not limited to the user's home, and may be a medical facility such as a designated hospital.

Next, a hardware configuration of the information processing apparatus 14 and the user terminal 12 according to the present embodiment will be described with reference to FIG. 2. As illustrated in FIG. 2, the information processing apparatus 14 includes a central processing unit (CPU) 20, a memory 21, a storage unit 22, a display 23, an input device 24, and a network interface (I/F) 25. The user terminal 12 includes a CPU 120, a memory 121, a storage unit 122, a display 123, an input device 124, a network I/F 125, and a Global Positioning System (GPS) 126.

The CPU 20 and the CPU 120 causes each device to realize a functional configuration to be described below based on a program stored in the storage unit 22 or the storage unit 122 to be described below. These CPUs are examples of a “processor” in the present embodiment.

The memory 21 and the memory 121 are memories for primary storage, and are, for example, RAMs such as a static random access memory (SRAM) or a dynamic random access memory (DRAM).

The storage unit 22 and the storage unit 122 are non-volatile memories, and indicate at least any one of, for example, a hard disk drive (HDD), a solid state drive (SSD), or a flash memory. An information processing program 30 is stored in the storage unit 22 as a storage medium. The CPU 20 reads out the information processing program 30 from the storage unit 22, loads the information processing program into the memory 21, and executes the loaded information processing program 30.

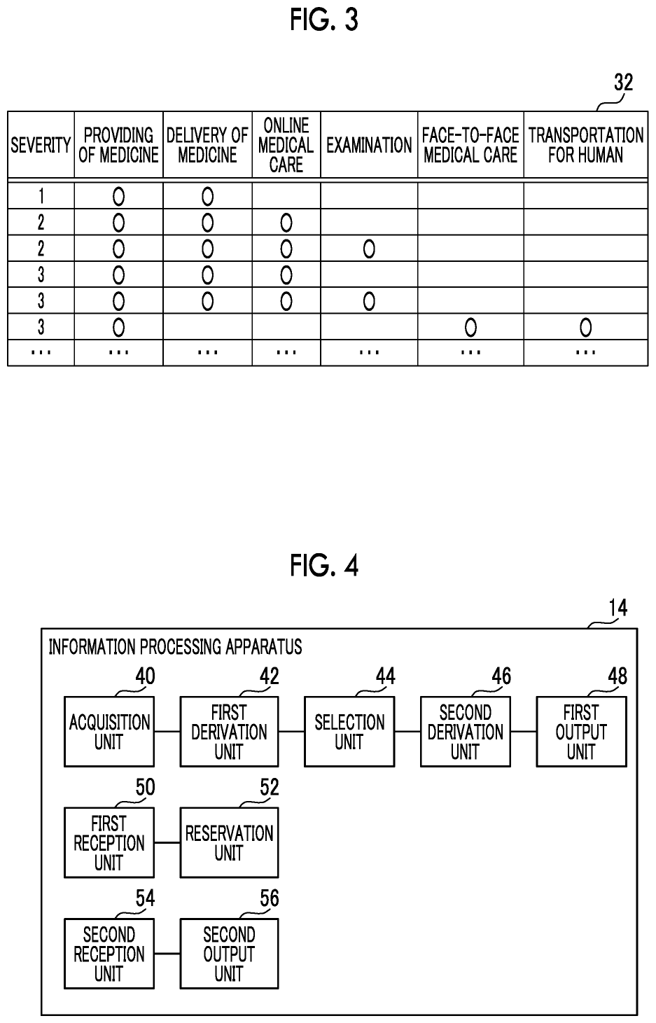

The storage unit 22 stores a service table 32 in which a severity is associated with at least one service. In the present embodiment, the severity is an integer value from 1 to a preset upper limit value, and the larger the value, the higher the severity. As illustrated in FIG. 3, in the service table 32, the service associated with the severity is stored for each severity. In the example of FIG. 3, a service marked with a circle is associated with a severity. For example, a set of the providing service for the medicine or the like and the delivery service for the medicine or the like is associated with a severity of “1”. A plurality of combinations of different services may be associated with one severity. In the example of FIG. 3, a set of the providing service for the medicine or the like, the delivery service for the medicine or the like, and the online medical care service and a set of the providing service for the medicine or the like, the delivery service for the medicine or the like, the online medical care service, and the examination service are associated with a severity of “2”.

The storage unit 122 stores a screen display program 130, and stores a program for displaying a display screen such as a questionnaire display screen and an action plan display screen to be described below on the display 123 based on data received from the information processing apparatus 14 via the network I/F 125 to be described below. The screen display program 130 indicates, for example, a program of a web browser or the like.

The display 23 and the display 123 indicate, for example, a liquid crystal display or an electroluminescent (EL) display. The display 23 displays an edit screen of the service table 32. The display 123 receives and displays an output of an action plan generated by the information processing apparatus 14 to be described below.

The input device 24 and the input device 124 indicate, for example, at least one of a keyboard or a mouse. The input device 24 and the input device 124 are not limited thereto, and may be at least any one of a voice input device using a microphone or the like, a gesture input device using a camera or the like, or a non-contact input device such as a touch panel, or a combination thereof. For example, the input device 24 receives an edit input of the service table. The input device 124 receives selection processing of the action plan or the like by the user.

The network I/F 25 and the network I/F 125 execute information transmission between the information processing apparatus 14 and the user terminal 12 via the network N.

A bus 27 connects the CPU 20, the memory 21, the storage unit 22, the display 23, the input device 24, and the network I/F 25. The bus 127 connects the CPU 120, the memory 121, the storage unit 122, the display 123, the input device 124, the network I/F 125, and the GPS 126. The user terminal 12 includes the GPS 126.

Next, a functional configuration of the information processing apparatus 14 according to the present embodiment will be described with reference to FIG. 4. As illustrated in FIG. 4, the information processing apparatus 14 includes an acquisition unit 40, a first derivation unit 42, a selection unit 44, a second derivation unit 46, a first output unit 48, a first reception unit 50, a reservation unit 52, a second reception unit 54, and a second output unit 56. The CPU 20 executes the information processing program 30, and thus, the CPU functions as the acquisition unit 40, the first derivation unit 42, the selection unit 44, the second derivation unit 46, the first output unit 48, the first reception unit 50, the reservation unit 52, the second reception unit 54, and the second output unit 56.

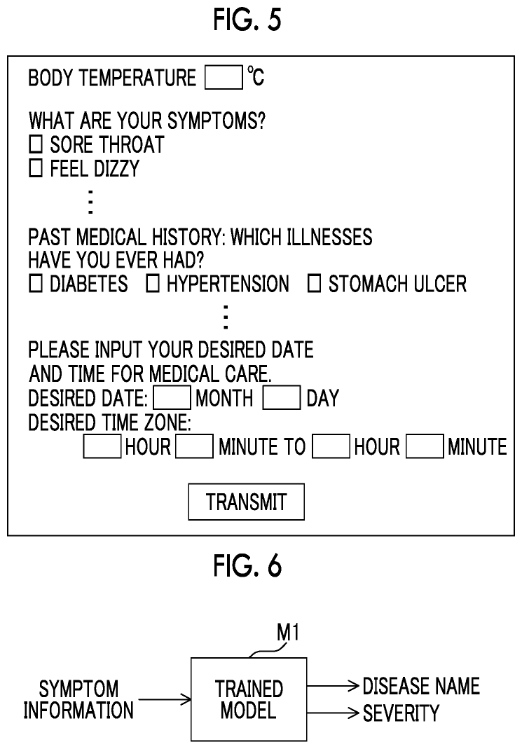

The acquisition unit 40 acquires symptom information on the symptom of the user transmitted from the user terminal 12, positional information, and desired date and time information including a date and a time zone in which the user desires to provide the service. For example, as illustrated in FIG. 5, the questionnaire display screen is displayed on the display 123 by loading the screen display program 130 stored in the storage unit 122 of the user terminal 12 into the memory 121 and executing the screen display program of the memory 121 by the CPU 120. The user inputs the symptom information and the desired date and time information to the questionnaire display screen via the input device 124. In the example of FIG. 5, the symptom information includes a body temperature of the user, a subjective symptom, and a past history. In a case where the user designates a transmission button after the symptom information and the desired date and time information are input on the questionnaire display screen, the symptom information, the positional information, and the desired date and time information are transmitted from the user terminal 12 to the information processing apparatus 14 via the network I/F 125.

User provision information transmitted from the user terminal 12 is not limited thereto. For example, the information processing apparatus 14 may acquire desired positional information indicating a place or the like where the service is desired to be provided. The information processing apparatus 14 may determine a priority of the service of the action plan to be described below or a delivery destination of the delivery service based on the acquired desired positional information. Information on a medical insurance company to which the user subscribes may be added to the user provision information. In a case where the face-to-face medical care service and the online medical care service of the action plan to be described below are selected, the information processing apparatus 14 may extract only hospitals in which the medical insurance of the medical insurance company can be used, or may determine a priority of each service of the face-to-face medical care service and the online medical care service.

The symptom information may include biological information of the user such as a heart rate measured by a wearable device such as a smart watch worn by the user in daily life. The positional information may be transmitted from the user terminal 12 based on Global Navigation Satellite System (GNSS) mounted on the user terminal 12. An example of the GNSS is Global Positioning System (GPS). The positional information may be transmitted from the user terminal 12 based on an address input by the user on the questionnaire display screen.

The first derivation unit 42 derives an estimated disease name and a severity of the user based on the symptom information acquired by the acquisition unit 40. Specifically, as illustrated in FIG. 6 as an example, the first derivation unit 42 derives the disease name and the severity by inputting the symptom information into a trained model M1. The trained model M1 is constructed by, for example, a neural network in which the symptom information is input and the disease name and the severity are output. The trained model M1 is, for example, a model trained by machine learning using, as training data, a large number of combinations of the symptom information and the disease name and the severity corresponding to the symptom information.

The selection unit 44 selects a service to be used by the user based on the severity derived by the first derivation unit 42. Specifically, the selection unit 44 refers to the service table 32 and selects the service associated with the severity derived by the first derivation unit 42.

The second derivation unit 46 derives an action plan of the user including a schedule and a service to be used by the user among a plurality of services related to medical care, based on the service selected by the selection unit 44 and the positional information acquired by the acquisition unit 40. Specifically, the second derivation unit 46 inquires of the service providing apparatus 16 for the service selected by the selection unit 44 about availability in a desired date and a desired time zone indicated by the desired date and time information acquired by the acquisition unit 40. The second derivation unit 46 derives the action plan of the user based on the availability of the service selected by the selection unit 44. This action plan is derived to be scheduled such that the user can use the service selected by the selection unit 44 in the desired time zone of the desired date. In a case where there are a plurality of combinations of services selected by the selection unit 44, the second derivation unit 46 derives the action plan of the user for each combination of the plurality of services.

In a case where the service selected by the selection unit 44 includes the online medical care service, the face-to-face medical care service, or the examination service, the second derivation unit 46 may inquire about availabilities in a medical care department and an examination apparatus corresponding to the disease name derived by the first derivation unit 42.

In a case where there are the plurality of combinations of services selected by the selection unit 44, the second derivation unit 46 derives a priority for displaying the action plan based on a selection history of the user for the service included in the action plan for each of the derived plurality of action plans. For example, the second derivation unit 46 raises the priority as a total value of the number of past selections of the service included in the action plan by the user is larger. The number of selections of the user in this case may be the number of selections of a derivation target user of a current action plan, or the number of selections of a group of users having the same symptom as the derivation target user. The number of selections of the user in this case may be the number of selections of a group of users having the same personal data (for example, age, gender, or the like) as the derivation target user, or the number of selections of all the users of the information processing system 10.

The second derivation unit 46 derives an assumed cost to be incurred in a case where the user acts according to the derived action plan. For example, in the online medical care service, the face-to-face medical care service, the examination service, and the providing service for the medicine, a standard cost is set in advance in accordance with the combination of the disease name and the severity derived by the first derivation unit 42. For example, a standard cost per unit distance is set in advance for the delivery service for the medicine and the transportation service for the human.

The second derivation unit 46 derives, as the assumed cost, the standard cost corresponding to the combination of the disease name and the severity for the online medical care service, the face-to-face medical care service, the examination service, and the providing service for the medicine. The second derivation unit 46 derives the assumed cost for the delivery service for the medicine and the transportation service for the human by multiplying a medicine delivery distance and a human movement distance by the standard cost per unit distance.

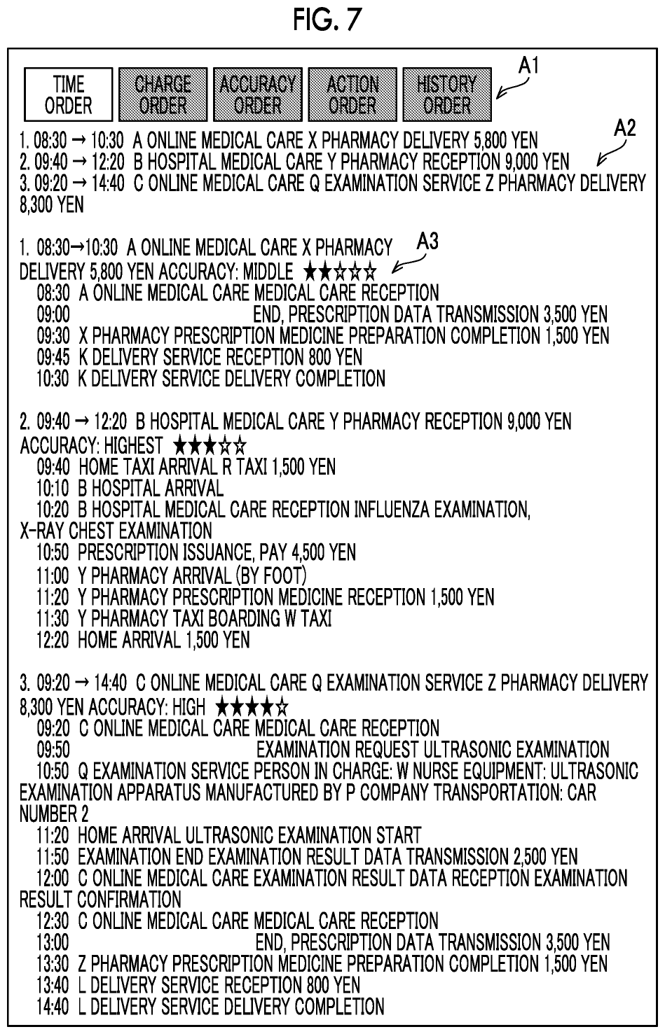

The first output unit 48 outputs (that is, transmits) display data including the action plan, the assumed cost, and the priority derived by the second derivation unit 46 to the user terminal 12. This display data is output in a data format that can be displayed on the display of the user terminal 12, such as a HyperText Markup Language (HTML) format. In a case where the display data transmitted from the information processing apparatus 14 is received, the user terminal 12 displays the action plan and the assumed cost included in the display data on the display 123. FIG. 7 illustrates an example of the action plan display screen displayed on the display 123 of the user terminal 12.

As illustrated in FIG. 7, the action plan display screen includes three display regions A1 to A3. In the first display region A1, a button for the user to designate a display order of the action plan is displayed. In the example of FIG. 7, the user can designate five orders of a time order, a charge order, an accuracy order, an action order, and a history order, and the time order is designated. The time order means the order in which an end time point is earlier. The charge order means the order in which a total amount of assumed costs is low. The accuracy order means the order in which the accuracy of medical care is high. The accuracy of medical care referred to herein means that the accuracy of medical care decreases in the order of the face-to-face medical care, the online medical care+examination, only the online medical care, and only the delivery for the medicine. The action order means the order in which the movement distance of the user is short. The history order means the order in which the priority derived by the second derivation unit 46 described above is high. That is, the second derivation unit 46 may derive the priority so that the time order, the charge order, the accuracy order, or the action order can be displayed.

An outline of the action plan is displayed in the second display region A2. In the example of FIG. 7, the outline of the action plan includes a scheduled time at which the user starts an action, a scheduled time at which the user ends the action, a service to be used by the user, and a total amount of assumed costs.

Details of the action plan are displayed in the third display region A3. In the example of FIG. 7, the service used in the action plan derived for each combination of three services and the assumed cost of the service are displayed from the top in the order in which the user uses the service. For example, the first action plan of FIG. 7 represents that the reception of the online medical care is started at 8:30 and the reception of the online medical care is completed at 9:00. The first action plan of FIG. 7 represents that prescription data issued by the doctor who performs the online medical care at 9:00 is transmitted to the pharmacy and the preparation of the medicine based on the prescription is completed at the pharmacy at 9:30. The first action plan of FIG. 7 represents that a delivery person who delivers the medicine at 9:45 receives the medicine from the pharmacy, the delivery of the medicine to the user is completed at 10:30, and the action plan is completed. The prescription data may be transmitted to the pharmacy through the information processing system 10. The information processing system 10 detects that the online medical care is ended by receiving the prescription data.

The second action plan of FIG. 7 represents that a taxi is dispatched to the user's home at 9:40, and the user gets on the taxi and heads for the hospital for face-to-face medical care. In the second action plan of FIG. 7, the user arrives at the hospital at 10:10, receives the medical care and examination at 10:20, listens to an explanation for the diagnosis and prescription at 10:50, and receives an issued prescription. The prescription may be received directly by the user, or the prescription data may be transmitted by the doctor through the pharmacy or the information processing system 10. In the second action plan of FIG. 7, the user heads to the pharmacy at 11:00, receives prescription medicine at 11:20, and heads home by the taxi delivered to the pharmacy at 11:30.

In the third action plan of FIG. 7, the user receives an online medical care at home at 9:20. The doctor in the online medical care notifies the examination service of the ultrasonic examination request after the medical care reception, and the examination service receives the request notification and determines to execute the examiner, the examination apparatus, and the delivery necessary for the examination reserved in advance. Then, the examiner gets on the delivery car together with the examination apparatus and heads to the user's home. In the third action plan of FIG. 7, an examiner of the examination service visits the user's home at 11:20 and examines the user. After the end of the examination, the examiner transmits the examination data to the information processing system 10. In receiving the examination data, the information processing system 10 detects that the examination action is ended and transmits the examination data to the online medical care service. The doctor of the online medical care service who received the examination data makes a diagnosis based on the examination data. In the third action plan of FIG. 7, at 12:30, the user receives an online medical care again and receives an explanation of the examination result, the diagnosis, and the prescription. After that, the doctor transmits the prescription data to the pharmacy via the information processing system 10, and the pharmacy delivers the prescription medicine dispensed based on the prescription data to the user's home.

The assumed cost of the service is displayed on a line corresponding to each service in the display region A3. In the example of FIG. 7, an outline of the action plan, the accuracy of the medical care, and a recommendation level are displayed on a first line of each action plan in the display region A3. In the example of FIG. 7, the recommendation level is represented by the number of star marks filled in black. This recommendation level is derived based on public data such as user reviews.

Actions represented in the action plan are not limited to the above examples. For example, the action plan can be made even in a case where the user receives an examination by a taxi toward an examination service in order to perform an examination in which medical diagnostic equipment is difficult to move such as a CT examination and an MRI examination, the doctor visits the user's home by a delivery vehicle and diagnoses the user in a case where the user cannot move, and heads to a hospital in which the examination service using the medical diagnostic equipment is the face-to-face medical care service for the examination in the medical diagnostic equipment not possessed by the hospital of the face-to-face medical care service.

Regarding the delivery service for the medicine or the like, medical supplementary products (for example, oral rehydration solutions, dressings, masks, disinfectants, diapers, and the like) that are sold in the pharmacy are recommended based on the symptom information of the user, and the medicine may be delivered together with the medicine in a case where the user approves.

The user selects a desired action plan from among the action plans displayed on the display of the user terminal 12. The first reception unit 50 receives the action plan selected by the user.

The reservation unit 52 executes reservation processing of the service used in the action plan received by the first reception unit 50. Specifically, the reservation unit 52 transmits reservation information for reserving such that the service is to be used according to the schedule included in the action plan to the service providing apparatus 16 for each service included in the action plan received by the first reception unit 50.

The second reception unit 54 receives billing information of the cost from each service used by the user after the user uses the service based on the action plan received by the first reception unit 50. This billing information is transmitted from the service providing apparatus 16 for each service to the information processing apparatus 14.

In order for the information processing system 10 to receive the billing information, service implementation information that supports the fact that the service is actually implemented may be acquired. The service implementation information may be acquired by the information processing apparatus 14, or may be acquired by each service providing apparatus 16 and may be transmitted to the information processing apparatus 14. For example, in the online medical care service, it may be determined whether or not the online medical care is implemented by acquiring electronic medical record information or network connection information as the service implementation information by the information processing system 10 and determining whether or not there is electronic medical record information or network connection information corresponding to an online medical care time by the information processing system 10. In the delivery service for the medical practice, a user who requests the delivery, an examiner and a doctor who are delivery targets, or a driver of a vehicle for delivery may transmit, as the service implementation information, information indicating the completion of the boarding, alighting, or delivering of the vehicle for delivery to the information processing system 10, and the information processing system 10 determines whether or not the implementation of the delivery service for the reserved medical practice is completed based on the acquired service implementation information. The information indicating the completion of the delivery includes, for example, authentication information indicating that the user receives the medicine in a case where the medicine is received from the delivery person of the medicine (for example, a signature, information transmission and reception by personal information between information terminals, or the like) in the case of the delivery service for the medicine or the like. In the delivery service for the medical practice, the information processing system 10 may acquire, as the service implementation information, GPS information of the vehicle for delivery, and the information processing system 10 may determine whether or not the delivery service for the reserved medical practice is implemented depending on whether or not the acquired GPS information of the vehicle for delivery satisfies positional information between two points of reserved delivery source and delivery destination. In the face-to-face medical care service, the examination service, or the providing service for the medicine or the like, an electronic medical record, an examination result, and delivery information of the medicine or the like may be input, as the service implementation information, to each service providing apparatus 16, and the information processing apparatus 14 receives the service implementation information transmitted from the service providing apparatus 16. As a result, it may be determined whether or not the implementation of each service is completed according to the reserved information.

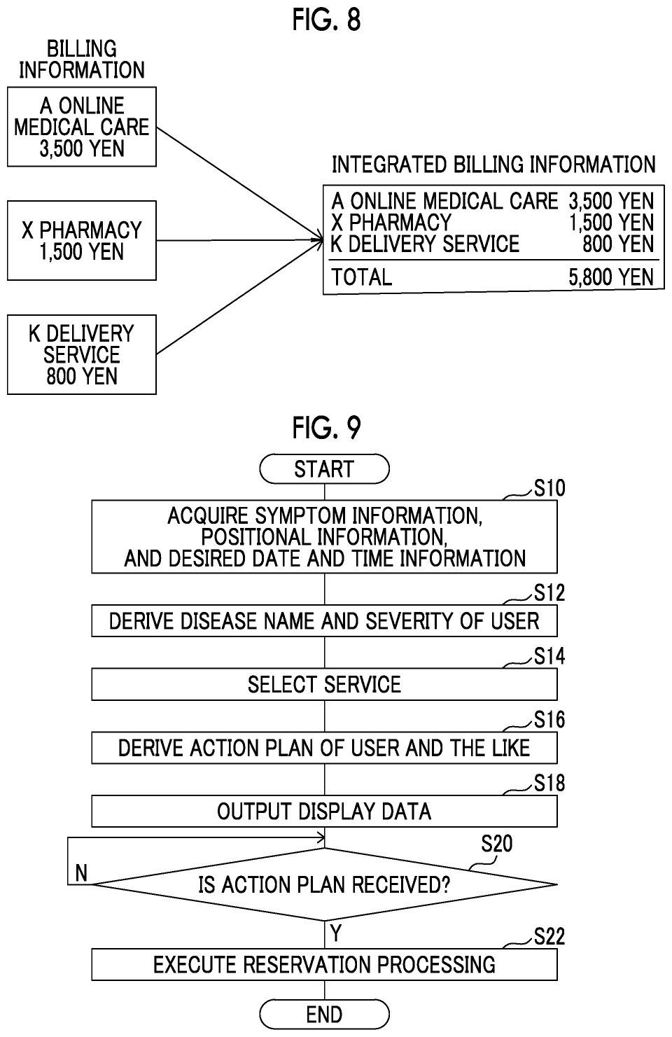

As illustrated in FIG. 8, as an example, the second output unit 56 generates integrated billing information in which the billing information received by the second reception unit 54 is integrated, and outputs the generated integrated billing information to the user terminal 12. In the example of FIG. 8, the integrated billing information in a case where the user uses the online medical care service, the providing service for the medicine, and the delivery service for the medicine is illustrated. The integrated billing information includes billing information for each service and a total amount of costs billed from each service. The user terminal 12 receives the integrated billing information transmitted from the information processing apparatus 14. The user refers to the integrated billing information and pays the cost. In this case, the information processing apparatus 14 may pay the cost for each service on behalf of the user. In this case, the user may pay the total amount included in the integrated billing information once.

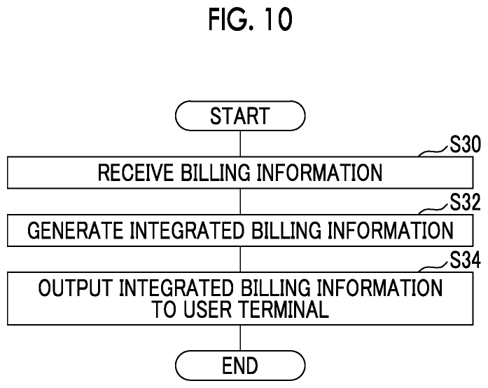

Next, an effect of the information processing apparatus 14 according to the present embodiment will be described with reference to FIGS. 9 and 10. The CPU 20 executes the information processing program 30 to execute action plan derivation processing illustrated in FIG. 9 and cost billing processing illustrated in FIG. 10. The action plan derivation processing illustrated in FIG. 9 is executed, for example, in a case where the information processing apparatus 14 receives the symptom information, the positional information, and the desired date and time information transmitted from the user terminal 12.

In step S10 of FIG. 9, the acquisition unit 40 acquires the symptom information, the positional information, and the desired date and time information transmitted from the user terminal 12. In step S12, as described above, the first derivation unit 42 derives the estimated disease name and the severity of the user based on the symptom information acquired in step S10. In step S14, as described above, the selection unit 44 refers to the service table 32 and selects the service to be used by the user based on the severity derived in step S12.

In step S16, as described above, the second derivation unit 46 derives the action plan of the user based on the service selected in step S14 and the positional information acquired in step S10. As described above, in a case where there are the plurality of combinations of services selected in step S14, the second derivation unit 46 derives the priority for displaying the action plan based on the selection history of the user for the service included in the action plan for each of the derived plurality of action plans. As described above, the second derivation unit 46 derives the assumed cost to be incurred in the case where the user acts according to the derived action plan.

In step S18, the first output unit 48 outputs display data including the action plan, the assumed cost, and the priority derived in step S16 to the user terminal 12. In step S20, the first reception unit 50 waits until the action plan is selected by the user on the action plan display screen displayed on the display of the user terminal 12 by the processing of step S18. In a case where the first reception unit 50 receives the action plan selected by the user, the determination in step S20 is a positive determination, and the processing proceeds to step S22.

In step S22, as described above, the reservation unit 52 executes the reservation processing of the service used in the action plan received in step S20. In a case where the processing of step S22 is ended, the action plan derivation processing is ended.

The cost billing processing illustrated in FIG. 10 is executed in a case where the information processing apparatus 14 receives the billing information of the cost from each service used by the user after the user uses the service based on the action plan selected by the above-described action plan derivation processing.

In step S30 of FIG. 10, the second reception unit 54 receives the billing information of the cost from each service used by the user. In step S32, the second output unit 56 generates the integrated billing information in which the billing information received in step S30 is integrated. In step S34, the second output unit 56 outputs the integrated billing information generated in step S32 to the user terminal 12. In a case where the processing of step S34 is ended, the cost billing processing is ended.

As described above, according to the present embodiment, it is possible to present an appropriate action plan regarding the medical care to the user.

In the above-described embodiment, the user can use the information processing system 10 at home or on an outing destination. For example, in a case where the user uses the service at an accommodation facility on the outing destination, even though the user is moving to the accommodation facility, the user inputs positional information of the accommodation facility as positional information of the user, and thus, the information processing system 10 can be used.

The user receives the online medical care in a car while the user is traveling a long distance on the Shinkansen or the like, inputs a boarding train name and an exit station, and designates, as a reception place of the medicine, places such as a boarding gate of a stop-over station, a pass box installed in the exit station, and a pharmacy adjacent to the exit station. As a result, even though the user is moving, the information processing system 10 can be used.

The user may be requested by the doctor to perform the re-examination for follow-up according to the diagnosis result. In this case, as a re-examination reservation, after the doctor and the user agree on medical care date and time, the hospital may transmit the medical care date and time of the re-examination to the information processing system 10, and the information processing system 10 may make the action plan using the medical care date and time as a reference and may transmit the action plan to the user. The information processing system 10 transmits action plan information of the re-examination or a link destination of the action plan to an e-mail address of the user entered in advance at the time of user registration, and the user approves the contents and reserves a re-examination plan.

In the above-described embodiment, the CPU 20 may notify the user of a timing of taking the medicine received by the user. In this case, for example, the CPU 20 transmits a message prompting the user to take the medicine to the user terminal 12 at a medicine taking timing determined for each medicine. In a case where a next medical care date is determined in the online medical care or the face-to-face medical care, the CPU 20 may notify the user of a next medical care timing. In this case, for example, the CPU 20 transmits a message indicating that the next day is the medical care date on the day before the medical care date to the user terminal 12.

In the above-described embodiment, the user terminal 12 may include each functional unit included in the information processing apparatus 14. In this case, the first output unit 48 outputs (that is, displays) the display data including the action plan, the assumed cost, and the priority derived by the second derivation unit 46 to the display of the user terminal 12.

In each of the above-described embodiment, the following various processors can be used as a hardware structure of processing units that execute various kinds of processing such as the acquisition unit 40, the first derivation unit 42, the selection unit 44, the second derivation unit 46, the first output unit 48, the first reception unit 50, the reservation unit 52, and the second reception unit 54 and the second output unit 56. As described above, in addition to the CPU which is a general-purpose processor that functions as various processing units by executing software (programs), the various processors include a programmable logic device (PLD), which is a processor capable of changing a circuit configuration after manufacture, such as a field programmable gate array (FPGA), and a dedicated electrical circuit, which is a processor having a circuit configuration specifically designed in order to execute specific processing such as an application specific integrated circuit (ASIC).

One processing unit may be constituted by one of these various processors, or may be constituted by a combination of two or more processors of the same type or different types (for example, a combination of a plurality of FPGAs or a combination of a CPU and an FPGA). The plurality of processing units may be constituted by one processor.

As an example in which the plurality of processing units are constituted by one processor, firstly, one processor is constituted by a combination of one or more CPUs and software as represented by computers such as clients and servers, and this processor functions as the plurality of processing units. Secondly, a processor that realizes the functions of the entire system including the plurality of processing units via one integrated circuit (IC) chip is used as represented by a system on chip (SoC). As described above, the various processing units are constituted by using one or more of the various processors as the hardware structure.

More specifically, an electric circuitry in which circuit elements such as semiconductor elements are combined can be used as the hardware structure of these various processors.

In the above-described embodiment, although the aspect in which the information processing program 30 is stored (installed) in the storage unit 22 in advance has been described, the present disclosure is not limited thereto. The information processing program 30 may be provided in a form of being recorded on a recording medium such as a compact disc read only memory (CD-ROM), a digital versatile disc read only memory (DVD-ROM), and a Universal Serial Bus (USB) memory. Further, the information processing program 30 may be in a form of being downloaded from an external device via a network.

What is claimed is:

1. An information processing apparatus comprising at least one processor,
wherein the processor is configured to:
derive an action plan of a user including a schedule and a service to be used by the user among a plurality of services related to medical care, based on symptom information and positional information related to a symptom of the user; and
output the derived action plan.

2. The information processing apparatus according to claim 1,
wherein the plurality of services include a service related to medical practice and a service related to delivery for the medical practice.

3. The information processing apparatus according to claim 2,
wherein the service related to the medical practice includes an online medical care service, a face-to-face medical care service, an examination service, and a providing service for medicine, and
the service related to the delivery includes a delivery service for medicine, a visit service for examination, and a transportation service for a human.

4. The information processing apparatus according to claim 1,
wherein the processor is configured to:
derive the action plan and an assumed cost to be incurred in a case where the user acts according to the action plan; and
output the derived assumed cost.

5. The information processing apparatus according to claim 1,
wherein the processor is configured to:
derive a severity of the user based on the symptom information; and
select the service to be used by the user based on the severity.

6. The information processing apparatus according to claim 1,
wherein the processor is configured to:
receive the action plan selected by the user; and
execute reservation processing of the service to be used in the received action plan.

7. The information processing apparatus according to claim 1,
wherein the processor is configured to:
receive billing information of a cost from each service used by the user after the user uses the service based on the action plan; and
output integrated billing information in which the received billing information is integrated.

8. The information processing apparatus according to claim 1,
wherein the processor is configured to:
derive a plurality of the action plans based on the symptom information and the positional information; and
output the derived plurality of the action plans.

9. The information processing apparatus according to claim 1,
wherein the processor is configured to:
derive a priority for displaying the action plan, based on a selection history of the user for the service included in the action plan; and
output the action plan and the derived priority.

10. An information processing method executed by a processor included in an information processing apparatus, the method comprising:
deriving an action plan of a user including a schedule and a service to be used by the user among a plurality of services related to medical care, based on symptom information and positional information related to a symptom of the user; and
outputting the derived action plan.

11. A non-transitory computer-readable storage medium storing an information processing program causing a processor included in an information processing apparatus to execute a process comprising:
deriving an action plan of a user including a schedule and a service to be used by the user among a plurality of services related to medical care, based on symptom information and positional information related to a symptom of the user; and
outputting the derived action plan.
